(19) United States
(12) Patent Application Publication
(10) Pub. No.: US 2015/0305703 A1
(43) Pub. Date: Oct. 29, 2015

(54) METHOD AND DEVICE FOR CONTROLLING ROTATION ANGLE OF C-ARM OF MEDICAL IMAGING APPARATUS

(71) Applicant: CORELINE SOFT CO., LTD., Seoul (KR)

(72) Inventors: Jin Kook Kim, Seoul (KR); Taek Seo Won, Seongnam (KR)

(73) Assignee: CORELINE SOFT CO., LTD.

(21) Appl. No.: 14/698,019

(22) Filed: Apr. 28, 2015

(30) Foreign Application Priority Data
Apr. 28, 2014 (KR) 10-2014-0050921

(51) Int. Cl. A61B 6/00

(52) U.S. Cl. CPC A61B 6/547; A61B 6/4441; A61B 6/469; A61B 6/465; A61B 6/463

(57) ABSTRACT

A method and device for controlling a medical imaging apparatus are disclosed herein. The device for controlling a medical imaging apparatus includes a processor. The processor controls a medical imaging apparatus to take a plurality of images including a region of interest of a subject, while a C-arm of the medical imaging apparatus rotates from a first rotation angle to a second rotation angle. The processor detects the rotation angle of the C-arm of the medical imaging apparatus corresponding to each of the taken images. The processor stores the detected rotation angle in a database, along with the taken image. The processor extracts a rotation angle, stored along with an image selected as an optimal image from the taken images, as an optimal rotation angle.

### CROSS-REFERENCE TO RELATED APPLICATION

This application claims under 35 U.S.C. §119(a) the benefit of Korean Application No. 10-2014-0050921 filed Apr. 28, 2014, which is incorporated herein by reference.

### TECHNICAL FIELD

Embodiments of the present invention relate, in general, to a method and device for controlling the rotation angle of a C-arm of the medical imaging apparatus and, more particularly, to technology for searching for the rotation angle of a C-arm at which an optimal medical image can be acquired and controlling the C-arm so that the C-mm has an optimal rotation angle.

### BACKGROUND ART

Different types of medical imaging and diagnosing apparatuses are used to diagnose affected parts of patients or subjects who visit hospitals. Among these apparatuses, an X-ray radiography apparatus is most widely used.

In general, an X-ray imaging apparatus shows the differences in absorptivity between different internal organs of a human body using X-rays, the degree of absorption of which varies depending on material, in the form of an X-ray image (a shadow image acquired by projecting different internal structures of a human body onto a film). The X-ray imaging apparatus is used not only for the purpose of the medical diagnosis of diseases of the large intestine, stomach ailments, tuberculosis or the like but also for the purpose of the non-destructive inspection of a structure for an internal defect.

The X-ray imaging apparatus includes an X-ray source for emitting X-ray and a detector for absorbing or detecting X-rays transmitted through a patient or a subject. With the development of technology, such a detector has been evolved into a film, a CCD, a CMOS image sensor, etc.

A C-arm has been used in X-ray imaging apparatuses in order to make it possible to adjust the imaging direction of an X-ray source and a detector depending on the posture, disease or affected part of a patient or a subject. The C-arm has a structure in which the X-ray source and the detector are installed on a C-shaped arc. The X-ray source and the detector are positioned at opposite ends within the C-arm so that a patient or a subject can be interposed between the X-ray source and the detector.

The arc of the C-arm is slidingly movable. Therefore, when the sliding rotation angle of the C-arm is adjusted, it is possible to acquire X-ray images at different angles with a patient or a subject kept in a fastened state.

A related technology for adjusting the rotation angle of the C-arm is disclosed in U.S. Pat. No. 7,175,346 entitled “Motorized Adjustable X-ray Apparatus” (issued on Feb. 13, 2007). This related technology introduces a configuration in which two factors, i.e., an angulation angle and an orbital angle, are adjustable and the motion of a C-arm is adaptively controlled. In this related technology, the orbital angle refers to a rotation angle based on the sliding of the C-arm, and the angulation angle refers to an angle based on the rotation of the C-arm itself.

Using a C-arm control technology, such as the above-described related technology, it is necessary in a hospital to obtain an angle used to acquire a highly optimized image for the diagnosis of a patient or a subject. Since the process of acquiring an optimized image is not automated, an operator determines a final rotation angle while adjusting the sliding rotation angle of the C-arm several times. In this process, a patient or a subject is cumulatively exposed to radiation. Accordingly, there is an urgent need for an effort to reduce the radiation exposure dose of a patient or a subject.

Among the medical imaging apparatuses capable of taking medical images, a fluoroscope which takes an X-ray moving image in real time is often denoted by a symbol, such as XA, in the field of radiology. Fluoroscopes usually have a C-arm structure. It will be apparent that the C-arm structure is not limited to the fluoroscope but may be utilized in other X-ray imaging apparatuses, such as a digital radiography apparatus.

In a medical imaging apparatus having a C-arm structure, an imaging angle with respect to a patient or a subject can be controlled by finely adjusting the sliding rotation angle of a C-arm. By doing so, it is made possible to obtain an optimized medical image suitable for the diagnosis of a patient or a subject. Conventionally, a method of taking X-ray images while repeatedly rotating a C-arm several times has been used in order to find the optimal sliding rotation angle of a C-arm. This is problematic in that the radiation exposure dose of a patient or a subject is increased.

### SUMMARY OF THE DISCLOSURE

Accordingly, the present invention has been made keeping in mind the above problems occurring in the prior art, and an object of the present invention is to provide a method and device for controlling a medical imaging apparatus, which are capable of optimizing the rotation angle of a C-arm.

More specifically, an object of the present invention is to provide a method and device for controlling a medical imaging apparatus, which are capable of detecting the rotation angle of a C-arm, taking images while rotating the C-arm, storing each of the taken images and the detected rotation angle together, and controlling the C-arm based on the stored angle so that the C-arm has an optimal angle.

In order to achieve the above objects, a method of controlling a medical imaging apparatus according to an embodiment of the present invention stores each of the frames of medical images, taken during the rotation of a C-arm, together with a C-arm sliding rotation angle, selects an optimal image candidate frame based on the taken images, and extracts the C-arm sliding rotation angle, stored along with the optimal image candidate frame, as an optimal rotation angle of the C-arm.

The control method may be implemented by a processor within a computing system or a processor within the medical imaging apparatus. The control method may be described in the form of program instructions, and may be loaded into and executed on memory connected to the processor.

In accordance with an aspect of the present invention, there is provided a method of controlling a medical imaging apparatus, including controlling a medical imaging apparatus so that the medical imaging apparatus takes a plurality of images, including a region of interest of a subject, while the C-arm of the medical imaging apparatus is rotating from a first rotation angle (a C-arm rotation start angle) to a second rotation angle (a C-arm rotation end angle); identifying the rotation angle of the C-arm of the medical imaging apparatus corresponding to each of the taken images; storing the identified rotation angle, together with the taken image; and extracting a rotation angle, stored along with an image selected as an optimal image from the taken images, as an optimal rotation angle.

In this case, the method may further include controlling the C-arm so that the C-arm is placed at the extracted rotation angle.

The method may further include providing a user menu which enables a user to select the optimal image from the taken images; and receiving a selection input related to the optimal image via the user menu.

Providing the user menu may include, if at least one of the taken images is displayed on a screen, displaying a rotation angle, stored along with the at least one image displayed on the screen, on the screen, along with the at least one image displayed on the screen.

Providing the user menu may include extracting a contour line of a pattern (a hole formed in a surgical operation part, an artifact attached for a special indication, or the like), predetermined with respect to the subject, from the at least one image displayed on the screen; and displaying the extracted contour line in such a way as to overlay the extracted contour line on the at least one image displayed on the screen.

Providing the user menu may include determining one or more optimal image candidates having highest suitability for the optimal image from the taken images based on the shape of a predetermined pattern with respect to the subject; and displaying the determined one or more optimal image candidates on a screen more prior to the other images (using a user interface capable of preferentially displaying the one or more optimal image candidates and allowing a user to select and confirm an optimal image).

The method may further include selecting the optimal image from the taken images based on a shape as which the region of interest of the subject is represented on the image. For example, in the method, a displayed image closest to a reference form in which the region of interest should be represented may be selected as the optimal image.

Selecting the optimal image may include searching for an image taken in a direction perpendicular to the region of interest, from the taken images based on the shape as which the region of interest of the subject is represented; and selecting the found image, taken in the direction perpendicular to the region of interest, as the optimal image.

In accordance with another aspect of the present invention, there is provided a device for controlling a medical imaging apparatus, including a processor which includes a few sub-modules; an imaging control unit configured to control a medical imaging apparatus so that the medical imaging apparatus takes a plurality of images, including a region of interest of a subject, while a C-arm of the medical imaging apparatus is rotating from a first rotation angle to a second rotation angle; a detection unit configured to detect the rotation angle of the C-arm of the medical imaging apparatus corresponding to each of the taken images; a storage control unit configured to store the detected rotation angle in a database, along with the taken image; and an extraction unit configured to extract a rotation angle, stored along with an image selected as an optimal image from the taken images, as an optimal rotation angle.

The device may further include a user interface control unit as another sub-module of the processor configured to provide a user menu that enables a user to select the optimal image from the taken images, and to receive a selection input related to the optimal image via the user menu.

The device may further include an image processing unit as another sub-module of the processor configured to search for an image, taken in a direction perpendicular to the region of interest, in the taken images while considering a form in which the region of interest of the subject is represented, and to select the found image as the optimal image.

According to the present invention, it is possible to easily acquire an optimized medical image suitable for the diagnosis of a patient or a subject using a C-arm type medical imaging apparatus. That is, the present invention makes it possible to search for an optimal sliding rotation angle based on images taken using a C-arm type medical imaging apparatus.

According to the present invention, it is possible to acquire medical images while rotating a C-arm within the specific range of C-arm sliding rotation angles, to search for an optimal image candidate through the processing of the medical images and the analysis of the characteristics thereof, and to extract an optimal C-arm sliding rotation angle from an imaging time-based sliding rotation angle stored along with the optimal image candidate.

This makes it possible to shorten the time required to search for the optimal C-arm sliding rotation angle and to simplify the process of searching for the optimal C-arm sliding rotation angle. It is also possible to automate the process of controlling a C-arm such that the C-arm is placed at the optimal sliding rotation angle.

According to the present invention, it is possible to minimize the imaging frequency or the imaging time required until a C-arm is placed at an optimal sliding rotation angle and to reduce the radiation exposure dose of a patient or a subject.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram illustrating a process of controlling a medical imaging apparatus according to an embodiment of the present invention;

FIG. 2 is an operation flowchart illustrating a method of controlling a medical imaging apparatus according to an embodiment of the present invention;

FIGS. 3 to 5 are operation flowcharts illustrating, in more detail, parts of the control method illustrated in FIG. 2;

FIG. 6 is a diagram illustrating a process of preferentially displaying an optimal image candidate in a process of controlling a medical imaging apparatus according to an embodiment of the present invention;

FIG. 7 is a block diagram illustrating a device for controlling a medical imaging apparatus according to an embodiment of the present invention; and

FIG. 8 is a block diagram illustrating a device for controlling a medical imaging apparatus according to another embodiment of the present invention.

### DETAILED DESCRIPTION OF THE DISCLOSURE

Embodiments of the present invention will now be described in detail with reference to the accompanying drawings. In the following description, detailed descriptions of known elements or functions that may unnecessarily make the gist of the present invention obscure will be omitted.

However, the present invention is not limited to the embodiments described below. The same reference numerals are used throughout the different drawings to designate the same elements.

The term “user” used herein may refer to an operator or a technician who operates a medical imaging apparatus, or may refer to a physician/clinician/medical doctor who conducts a medical treatment/operation using a medical imaging apparatus.

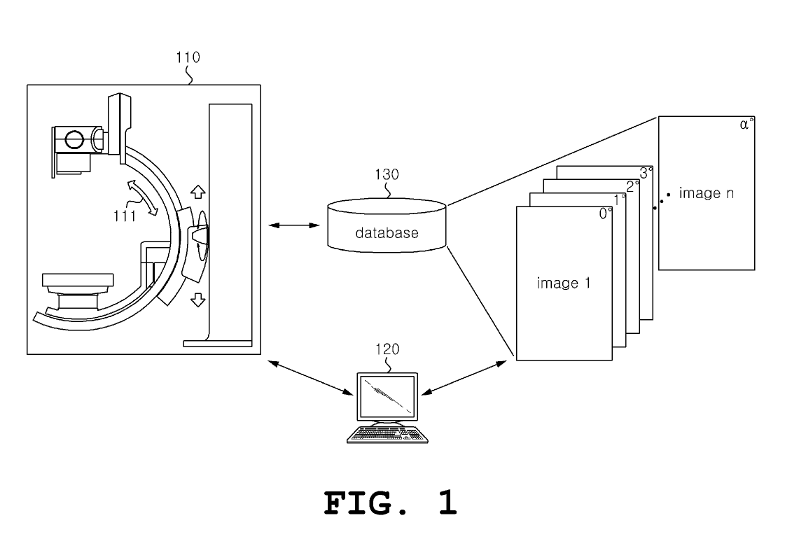

FIG. 1 is a diagram illustrating a process of controlling a medical imaging apparatus according to an embodiment of the present invention.

Referring to FIG. 1, a medical imaging apparatus 110 has a C-arm structure. The C-arm of the medical imaging apparatus 110 enables clockwise or counterclockwise sliding rotation 111. Since the orientation of an X-ray source and a detector is adjusted by the sliding rotation 111 of the C-arm, an imaging direction and an imaging angle during X-ray imaging are determined. The C-arm structure is widely used in fluoroscopes that take X-ray moving images in real time. The C-arm structure is also used in digital radiography apparatuses.

A device for controlling the medical imaging apparatus 110 according to an embodiment of the present invention may be implemented in the form of an internal processor of a computing system 120 or an internal processor of the medical imaging apparatus 110. The device for controlling the medical imaging apparatus 110 according to the embodiment of the present invention can determine the optimal angle of the C-arm sliding rotation 111 of the medical imaging apparatus 110 based on the medical images processed within the computing system 120. The device for controlling the medical imaging apparatus 110 according to the embodiment of the present invention may also control the C-arm sliding rotation angle of the medical imaging apparatus 110 in order to achieve the optimal sliding rotation angle determined as above.

The computing system 120 includes a processor, and a user interface, such as a display unit, a keyboard, a mouse or the like. The computing system 120 may display a medical image so that a user can identify the medical image. The processor of the computing system 120 may process or analyze the medical image as required.

A database 130 stores a medical image taken by the medical imaging apparatus 110. In this case, the database 130 may store information about the imaging time-based C-arm sliding rotation angle 111, together with the medical image taken by the medical imaging apparatus 110. Although the description made herein is focused on the C-arm sliding rotation angle 111 for convenience of description, the spirit of the present invention is not limited thereto. In another embodiment of the present invention, information about the height of the C-arm, the frame rotation angle or the like available at the imaging time may be additionally stored in the database 130 along with the medical images.

The C-arm sliding rotation angle 111 may be obtained via the use of an inertial sensor or an angle sensor, or may be obtained by regarding the control information of an actuator for controlling the C-arm sliding rotation angle 111 as the C-arm sliding rotation angle 111.

The medical imaging apparatus 110 acquires a series of medical images while sliding and rotating the C-arm within a specified angular range. In this case, when the medical imaging apparatus 110 is a fluoroscope, information about the imaging time-based C-arm sliding rotation angle 111 is transferred to the database 130 along with a medical image with respect to each frame of a taken moving image.

Each of the series of medical images may be acquired each time the C-arm slidingly rotates by a predetermined unit angle, or may be acquired on a predetermined unit time basis. In the case where the C-arm slidingly rotates at a constant angular velocity, the medical images acquired on a predetermined unit time basis will be acquired on a predetermined unit angle (sliding rotation) basis.

In a further embodiment of the present invention, a medical image may be taken at a predetermined rotation angle, and may be stored in the database 130 along with the rotation angle at which the medical image is taken.

The medical images of a patient or a subject are provided to a user (operator) via the computing system 120. The computing system 120 may also display information about the imaging time-based C-arm sliding rotation angles 111 stored in the database 130 along with the medical images of the patient or subject.

When the medical imaging apparatus 110 implemented in the form of the C-arm is a fluoroscope, the medical imaging apparatus 110 takes a moving image in real time, and provides the moving image in order to perform a medical treatment, such as angiography or the insertion of a needle for biopsy. In this case, the medical imaging apparatus 110 takes preliminary medical images. Among the preliminary medical images, an optimal present image candidate is selected by the control device included in the medical imaging apparatus 110 or the computing system 120. An optimal C-arm sliding rotation angle 111 at which a present medical image will be taken is derived using the selected present image candidate and information about the imaging time-based C-arm sliding rotation angles 111 stored in the database 130.

The control device may control the C-arm such that the C-arm is placed at the optimal C-arm sliding rotation angle 111 at which the present medical image will be taken. The present medical image is taken in the state in which the C-arm has been placed at the optimal C-arm sliding rotation angle 111 derived as above. The present medical image may be taken simultaneously with a medical treatment, such as angiography or the insertion of a needle for biopsy.

A process of placing the C-arm at the optical C-arm sliding rotation angle derived as above may be manually performed by a user (operator), may be performed by the control device (processor) installed within the computing system 120 or the medical imaging apparatus 110 according to the manipulation of a user, or may be automatically performed by the control device (processor) without intervention of a user.

The range of the C-arm sliding rotation angles 111 within which the preliminary medical images will be taken may be manually input by a user (operator), or may be determined by the control device (processor) of the computing system 120 or medical imaging apparatus 110 in order to conform to the purpose of the diagnosis or medical treatment of a patient or a subject. For example, if it is considered to be most advantageous based on common sense to take an image from a location immediately above a patient or a subject when a needle is inserted into the patient or the subject for the purpose of biopsy, the C-arm sliding rotation angle 111 may be set within a specific range around 90 degrees, and preliminary medical images may be taken at regular intervals of 1 degree or at regular time intervals (e.g., at intervals of 1 or 0.1 sec). In this case, depending on the posture of a patient or a subject or the position of a biopsy target organ, a value other than 90 degrees, e.g., 88 degrees, may be derived as the optimal C-arm sliding rotation angle. The present medical image acquired during biopsy is taken at the optimal C-arm sliding rotation angle. A process of selecting an optimal present image candidate from among the preliminary medical images may be achieved by the following embodiments. A first embodiment of the process of selecting a present image candidate may provide a user interface menu which enables a user (an operator or a technician) to select an optimal present image candidate. In this case, the user interface menu may be provided via the screen of the computing system 120. The computing system 120 may receive a selection input for an optimal present image candidate from a user via the user interface menu. In response to the selection input, the computing system 120 may provide the information about the imaging time-based C-arm sliding rotation angles stored in the database 130, together with the optimal present image candidate. Furthermore, when providing the user interface menu, the computing system 120 may display the information about the C-arm sliding rotation angles, stored along with the respective preliminary medical images, while displaying the preliminary medical images. For example, the computing system 120 may display the information about the imaging time-based C-arm sliding rotation angle in the right upper or lower end portion of each of the preliminary medical images.

A user can identify the C-arm sliding rotation angle at the time at which the optimal present image candidate selected by the user is taken. Accordingly, as described above, the C-arm may be positioned at the optimal sliding rotation angle via a user's manual operation, or the sliding rotation angle of the C-arm may be automatically adjusted by the control device of the present invention. In the case where a user manually positions the C-arm at the optimal sliding rotation angle, when the C-arm reaches the optimal sliding rotation angle during the user's manual adjustment of the C-arm sliding rotation angle, the arrival of the C-arm at the optimal sliding rotation angle may be displayed on the screen of the computing system 120 or may be notified using a sound of the computing system 120 or the medical imaging apparatus 110. Moreover, the arrival of the C-arm at the optimal sliding rotation angle may be notified to a user via the sensation of touch using the vibration of a specific part of the medical imaging apparatus 110. Alternatively, during the rotation of the C-arm of the medical imaging apparatus 110, the current C-arm sliding rotation angle may be displayed on the screen of the computing system 120. In this case, the C-arm sliding rotation angle needs to be detected using a sensor.

In a second embodiment of the process of selecting an optimal present image candidate from preliminary medical images, the image processing module of the computing system 120 may detect an image, predetermined by a user, from fluoroscopy images in which a C-arm sliding rotation angle has been stored with respect to each frame. The image predetermined by a user may include a predetermined pattern, such as a surgical operation part of a subject (a patient), a hole formed for the insertion of a needle, an artifact, or the like. The image processing module of the computing system 120 may automatically detect an image, predetermined by a user, from fluoroscopy images in which a C-arm sliding rotation angle has been store with respect to each frame. The computing system 120 may provide a user with an image of a frame, from which a predetermined image is detected, more preferentially than images of other frames. The term “preferentially provide” means that an image is conspicuously displayed so that a user can first see the image. One example of the preferential display may be displaying an image at the center of a screen in the largest size.

For this process, the image processing module of the computing system 120 may extract the contour line of an image part, corresponding to a pattern (a hole formed in a surgical operation part, an artifact attached for a special indication, etc.) predetermined with respect to a patient or a subject, from each of the preliminary medical images to be displayed on a screen. The computing system 120 may display the contour line of the pattern, extracted from each of the frames of the preliminary medical images, together with each of the preliminary medical images. A user should have an optimal pattern reference image predetermined with respect to the pattern. Accordingly, the user may select an image, having the highest coincidence (suitability) with the optimal pattern reference image, as an optimal preliminary medical image, i.e., an optimal present image candidate, based on the contour line of the displayed pattern.

The comparison of the contour line of the extracted pattern with the predetermined optimal pattern reference image may be also performed by the image processing module of the computing system 120. In this case, the image processing module of the computing system 120 may attempt to partially match the optimal pattern reference image against each of the frames of the preliminary medical images in order to search for an image part corresponding to the optimal pattern reference image. The computing system 120 most conspicuously (preferentially) displays the optimal present image candidate obtained through this process, thereby providing a user (an operator, a technician or an operating doctor) with an opportunity to identify the optimal present image candidate.

In this case, the optimal present image candidate may be selected depending on whether a region of interest (ROI) of a subject or a patient is perpendicular to an imaging direction or depending on the extent to which the region of interest (ROI) of a subject or a patient is placed in a vertical direction. That is, when the region of interest of a patient is perpendicular to the imaging direction, it is highly likely that the region of interest is imaged in the most easy-to-see form. Accordingly, the image processing module of the computing system 120 may search for an image frame, taken in a direction perpendicular to the region of interest, from the form in which the region of interest is placed in an image.

If the control device (processor) of the computing system 120 or the medical imaging apparatus 110 selects an image, taken in a direction perpendicular to a region of interest or in the direction closest to the perpendicular direction, as an optimal present image candidate, the C-arm sliding rotation angle may be controlled in order to become an angle perpendicular to the region of interest of a patient or a subject or an angle closest to a perpendicular angle. Accordingly, the present medical image may be taken at an angle perpendicular to a region of interest of a patient or a subject or an angle closest to a perpendicular angle.

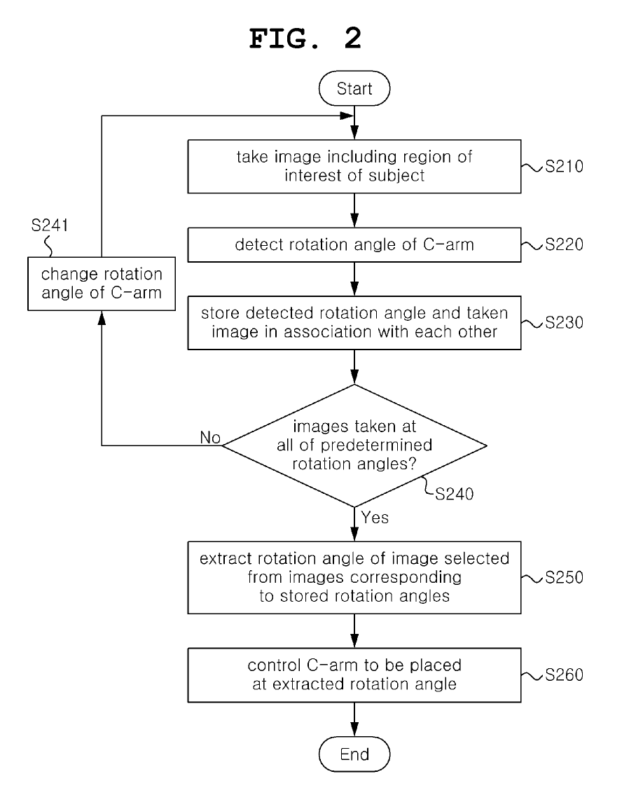

FIG. 2 is an operation flowchart illustrating a method of controlling the medical imaging apparatus 110 according to an embodiment of the present invention. More specifically, FIG. 2 is an operation flowchart illustrating a method of controlling the rotation angle of the C-arm of the medical imaging apparatus 110.

The method of controlling the medical imaging apparatus 110 illustrated in FIG. 2 may be implemented by the control device (or the processor; not illustrated) of the medical imaging apparatus 110 or the computing system 120.

Referring to FIG. 2, in the method of controlling the sliding rotation angle of the C-arm, a medical image including a region of interest of a patient or a subject is taken at step S210. In this case, the imaging time-based sliding rotation angle of the C-arm of the medical imaging apparatus 110 is detected at step S220. The region of interest may be an operation part, affected part or biopsy target part of a patient or a subject. Although step S220 has been illustrated after step S210 in FIG. 2, steps S210 and S220 may be simultaneously performed or step S220 may be performed prior to step S210 depending on the embodiment. In this case, a current C-arm sliding rotation angle is detected at step S220, in which state imaging is performed without changing the position of the C-arm at step S210.

In the method of controlling the medical imaging apparatus 110 according to the present invention, the detected rotation angle and the taken image including the region of interest of a subject are stored in association with each other at step S230. In this case, each frame of the taken medical images is stored in the database 130 along with the C-arm sliding rotation angle. Steps S210 to S230 are performed while the C-arm sliding rotation angle is being changed. In this case, it is determined whether the images have been taken at all of the predetermined C-arm sliding rotation angles at step S240. If any of the images has not been taken at the predetermined C-arm sliding rotation angles, steps S210 to S230 are repeated while the C-arm sliding rotation angle is being changed. At step S241, the C-arm sliding rotation angle may be changed in increments of a unit sliding rotation angle. The imaging and the detection of the angle, i.e., steps S210 to S230, may be performed at specific time intervals while the C-arm is being slidingly rotated. Meanwhile, although the predetermined sliding rotation angles may refer to a first rotation angle which is a C-arm rotation starting angle and a second rotation angle which is a C-arm rotation ending angle, the range of the rotation angles may be adjusted in accordance with the purpose of the diagnosis of a patient or a subject. As described above, the C-arm sliding rotation angle may be adjusted, for example, within the range of ±5 degrees around 60 degrees. In this case, 55 degrees, which is a rotation starting angle, may be referred to as a first rotation angle, and 65 degrees, which is a rotation ending angle, may be referred to as a second rotation angle. Moreover, the unit rotation angle may be an angle which is predetermined depending on the characteristics of C-arm equipment. A user may adjust the value of the unit rotation angle. In general, a minimum angle difference that can generate a meaningful difference between taken images may be set as the unit rotation angle.

If it is determined at step S240 that the C-arm has rotated from the first rotation angle to the second rotation angle and further that all the medical images have been taken at the predetermined sliding rotation angles, the rotation angle of the image selected by the user's input or the control device (processor) from the images corresponding to the stored rotation angles is extracted at step S250. The C-arm is controlled in order to be placed at the extracted rotation angle at step S260.

If it is determined at step S240 that all the medical images have not yet been taken at the predetermined sliding rotation angles, the sliding rotation angle of the C-arm is changed at step S241 and steps S210 to S230 are repeated.

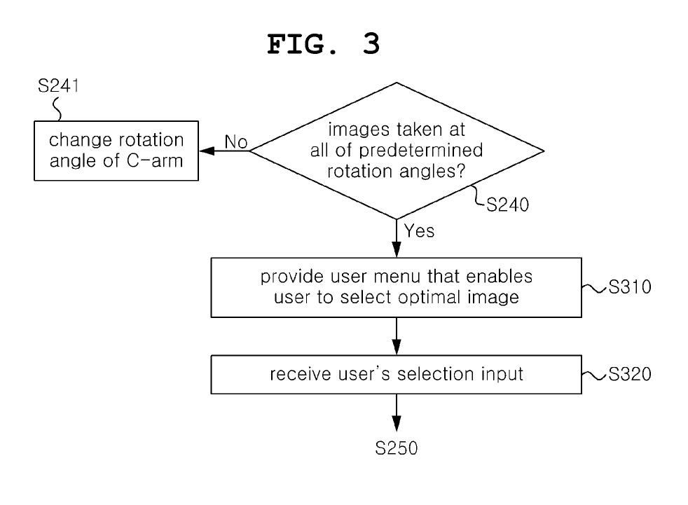

FIGS. 3 to 5 are operation flowcharts illustrating, in more detail, parts of the control method illustrated in FIG. 2. More specifically, FIGS. 3 to 5 illustrate various embodiments of a process of selecting an optimal image between steps S240 and S250. That is, between steps S240 and S250, the control device (processor) may display a user menu so that a user can directly select an optimal image candidate from the images taken at the respective rotation angles. This process is illustrated in FIG. 3. The user menu may be provided by the user interface (UI) of the computing system 120.

FIG. 3 is an operation flowchart illustrating one example of an optimal image selection process in the method of controlling the medical imaging apparatus 110 according to an embodiment of the present invention.

Referring to FIG. 3, if images have been taken at all of the predetermined C-arm sliding rotation angles at step S240, a user menu which enables a user to select an optimal image is provided via the user interface of the computing system 120 at step S310. The user interface of the computing system 120 receives a user's selection input relating to an optimal image at step S320.

Since steps S240 and S241 illustrated in FIG. 3 are identical in function with steps S240 and S241 illustrated in FIG. 2, descriptions thereof are omitted.

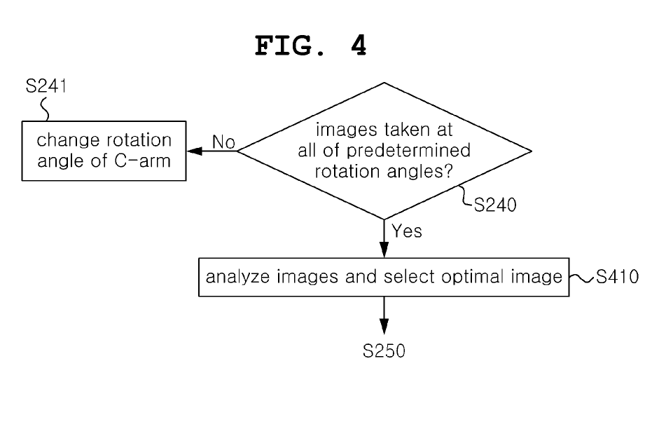

FIG. 4 is an operation flowchart illustrating one example of an optimal image selection process in the method of controlling the medical imaging apparatus 110 according to another embodiment of the present invention.

Referring to FIG. 4, if the images have been taken at all of the predetermined C-arm sliding rotation angles at step S240, the image processing module of the computing system 120 analyzes the taken medical images and selects an optimal image candidate at step S410.

The image processing module of the computing system 120 selects an optimal image candidate by analyzing the similarity between the predetermined optimal pattern reference image and each of the image frames. In this case, the image processing module may search for a part corresponding to a pattern (a hole formed in a surgical operation part, an artifact attached for a special indication, or the like) from the taken image frames.

The optimal pattern reference image may be set on the assumption that the image is taken in a direction perpendicular to the relevant pattern. This means that the optimal image candidate is selected such that the relevant pattern is represented on the medical image in the most easy-to-see form. That is, if the pattern is a region of interest (a surgical operation part or an affected part) designated by a user, the image taken in a direction perpendicular to the region of interest may be selected as the optimal image candidate such that the region of interest can be most conspicuously seen.

For example, in the case where a needle is inserted into the body of a patient in a medical treatment, the C-arm sliding rotation angle needs to be determined so that, when a present image is taken, the route of the needle can be easily identified via preliminary images. For this purpose, an imaging angle at which the pattern is most conspicuously seen may be estimated using a pattern, such as an artifact attached to the body of the patient, a biopsy target organ of the patent, or the trajectory of preliminary needle insertion conducted for the insertion of a needle.

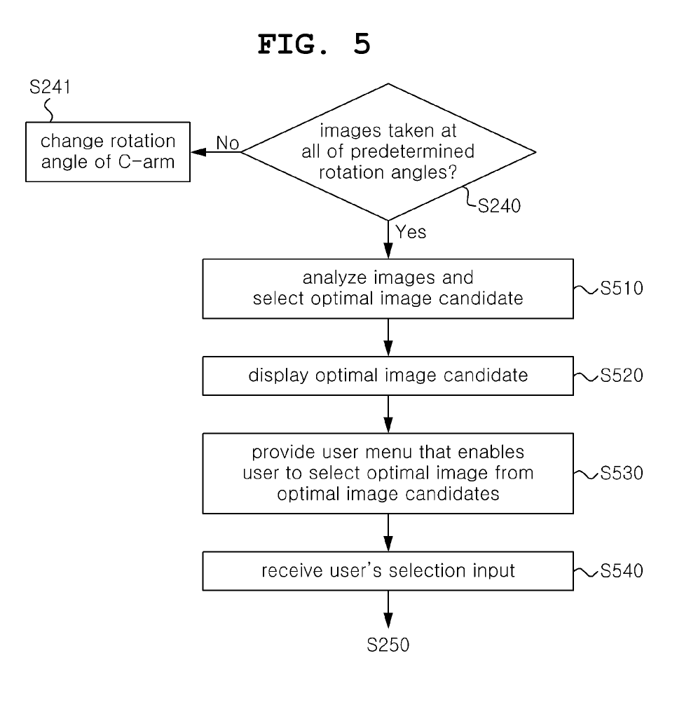

FIG. 5 is an operation flowchart illustrating one example of an optimal image selection process in the method of controlling the medical imaging apparatus 110 according to a further embodiment of the present invention.

Referring to FIG. 5, if images have been taken at all of the predetermined C-arm sliding rotation angles at step S240, the image processing module of the computing system 120 analyzes the taken images and selects one or more optimal image candidates at step S510.

The user interface module of the computing system 120 displays one or more optimal image candidates on a screen at step S520.

The user interface module of the computing system 120 provides a user menu so that a user can select an optimal image from the one or more optimal image candidates at step S530.

The user interface module of the computing system 120 receives a user's selection input relating to the optimal image at step S540.

In this case, at steps S520 and S530, the user interface module of the computing system 120 may display image frames in order from an image frame most suitable for the optimal image candidate, and may provide a user menu so that a user can easily select the optimal image candidate. The image most suitable for the optimal image candidate may be obtained by selecting an image most similar to a predetermined optimal pattern reference image using the image processing module of the computing system 120 or by searching for an image taken in a direction perpendicular to a region of interest.

The user interface module of the computing system 120 may most conspicuously display the image having the highest suitability. For example, the image having the highest suitability may be displayed at the center of a screen in the largest size. Alternatively, an image having the highest suitability among a series of images may be conspicuously expressed in a way that emphasizes a color, a size or a contour line.

While the user interface module of the computing system 120 preferentially displays the image having the highest suitability, it may be possible for the user interface module to provide, via the user menu, an option that enables a user to select another image as the optimal image candidate.

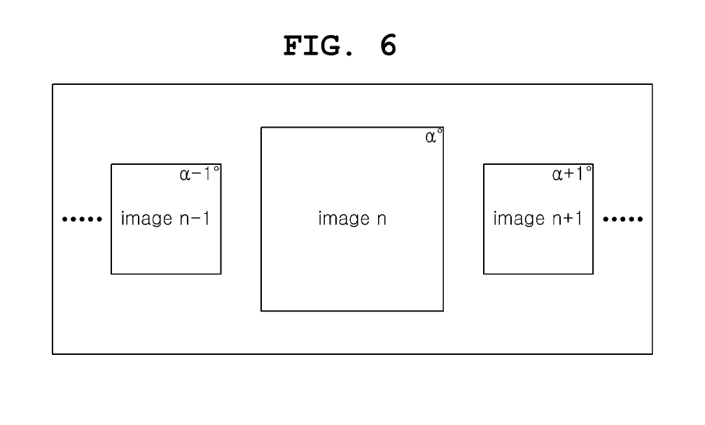

FIG. 6 is a diagram illustrating a process of preferentially displaying an optimal image candidate in a process of controlling the medical imaging apparatus according to an embodiment of the present invention.

Referring to FIG. 6, an image n having the highest priority selected as the optimal image candidate is displayed at the center of a screen in the largest size. Images n−1 and n+1 having the next priority may be displayed at the left and right sides of the image having the highest priority, and thus may be displayed along with the image having the highest priority.

In this case, detected C-arm sliding rotation angles (stored together in the database 130) may be displayed along with the images n, n−1 and n+1.

The images and the sliding rotation angles may be stored together in association with each other. The term “stored together” used herein is not limited to storing the images and the sliding rotation angles at the same addresses or at adjoining addresses. The storage of the images and the sliding rotation angles may be implemented in a way in which a sliding rotation angle corresponding to each of the images can be easily searched for in the database 130, such as a way in which the same indices are shared or the addresses of storage spaces are linked to each other.

While the user interface module of the computing system 120 preferentially provides a user with the image n, it may be possible to provide a user menu which enables a user to replace the preferentially displayed image so that an image other than the image n can be selected as the optimal image candidate according to the judgment of a user. For example, if a user clicks (or touches) the image n+1 illustrated in FIG. 6, the image n+1 may be displayed at the center of a screen in the largest size. If a user clicks (or touches) a confirmation menu option, the image n+1 may be selected as an optimal image candidate.

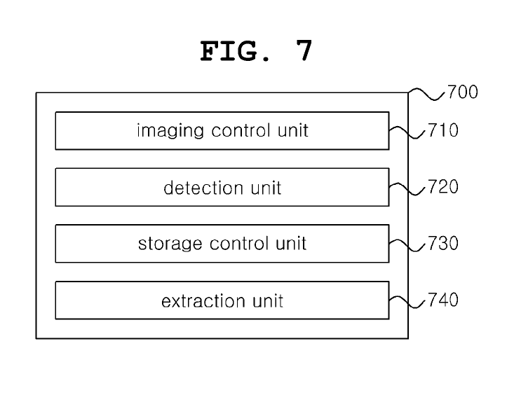

FIG. 7 is a block diagram illustrating a device 700 of controlling the medical imaging apparatus 110 according to an embodiment of the present invention. More specifically, FIG. 1 is a diagram illustrating an apparatus for controlling the sliding rotation angle of the C-arm of the medical imaging apparatus 110.

The control device 700 illustrated in FIG. 7 may be implemented in the form of a processor within the medical imaging apparatus 110 or the computing system 120. Meanwhile, the individual units 710 to 740 illustrated in FIG. 7 may be hardware modules which are present within the processor, or may be software modules which are implemented to execute specific functions.

The control device 700 includes an imaging control unit 710, a detection unit 720, a storage control unit 730, and an extraction unit 740. The imaging control unit 710 controls the medical imaging apparatus so that the medical imaging apparatus can take images including a region of interest of a subject at unit rotation angle intervals while the C-arm of the medical imaging apparatus rotates from a first rotation angle to a second rotation angle. The terms “first rotation angle” and “second rotation angle” used herein refer to angles at the start and end of the sliding rotation of the C-arm. The first rotation angle and the second rotation angle may be differently set depending on the purpose of imaging, diagnosis or medical treatment. For example, when the optimal sliding rotation angle of the C-arm is intuitively expected to be slightly smaller or larger than 90 degrees, the first rotation angle and the second rotation angle may be set within a small error range around 90 degrees. The unit rotation angle means a minimum sliding angle which is set such that a meaningful difference can be generated between the taken images. For example, when imaging is performed while the C-arm is slidingly rotating at intervals of 1 degree, the unit rotation angle can be considered to be 1 degree. In the case where imaging is performed at specific time intervals (e.g., intervals of 1 or 0.1 sec, or the like) while the C-arm is slidingly rotating, it will be necessary to detect an imaging time-based C-arm sliding rotation angle using a sensor (e.g., an inertial sensor, a rotation sensor, a sensor capable of measuring the rotation of a gear, or the like).

The detection unit 720 detects a C-arm rotation angle corresponding to each of the images taken at intervals of a unit rotation angle.

The storage control unit 730 stores the detected rotation angle, together with each of the taken images, in the database 130.

The extraction unit 740 extracts a rotation angle, stored in the database 130 along with an image selected as an optimal image from the taken images, as an optimal rotation angle.

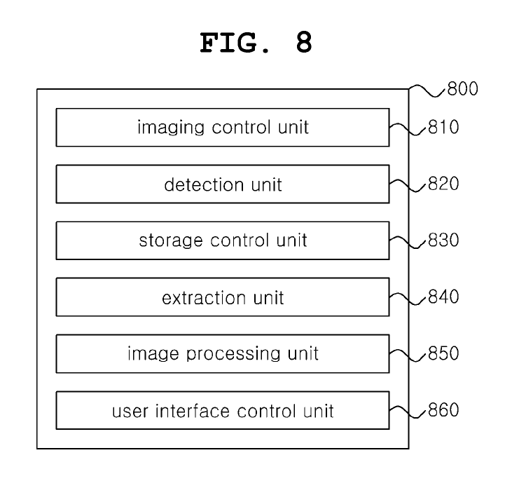

FIG. 8 is a block diagram illustrating a device 800 for controlling the medical imaging apparatus 110 according to another embodiment of the present invention.

The control device 800 illustrated in FIG. 8 may be implemented in the form of a processor (not illustrated) within the medical imaging apparatus 110, or may be implemented in the form of an internal processor (not illustrated) of the computing system 120 outside the medical imaging apparatus 110. The control device 800 may include an imaging control unit 810, a detection unit 820, a storage control unit 830, an extraction unit 840, an image processing unit 850, and a user interface control unit 860. Since the imaging control unit 810, the detection unit 820, the storage control unit 830, and the extraction unit 840 are identical in function with the imaging control unit 710, the detection unit 720, the storage control unit 730 and the extraction unit 740 illustrated in FIG. 7, descriptions thereof are omitted here.

Based on a form in which a region of interest of a subject is represented, the image processing unit 850 may search for an image, taken in a direction perpendicular to the region of interest, in the taken images, and may select the found image as an optimal image.

Furthermore, the user interface control unit 860 may provide a user menu which enables a user to select the optimal image from the taken images, and may receive an optimal image selection input via the user menu.

The image processing unit 850 and the user interface control unit 860 are illustrated together in FIG. 8. However, according to different embodiments of the present invention, an optimal image may be selected by the user interface control unit 860 without the use of the image processing unit 850, or may be selected by the function of the image processing unit 850 without the use of the user interface control unit 860.

An embodiment in which an optimal image is selected by the collaboration between the image processing unit 850 and the user interface control unit 860 may operate as follows. The image processing unit 850 may select one or more optimal image candidates from taken images depending on suitability for an optimal image. The user interface control unit 860 preferentially provides one or more optimal image candidates, thereby providing a user with an opportunity to preferentially examine one or more optimal image candidates. One or more optimal image candidates may be preferentially provided in such a way as to conspicuously express them on a screen using a method of emphasizing the size, color, or contours thereof. The one or more optimal image candidates may be preferentially provided via visual representation, and may be conspicuously expressed via an interface, such as the sensation of hearing (using voice or sound) or the sensation of touch sense (using vibration).

The present invention was derived from research conducted as a part of the Technological Innovation Development Project sponsored by the Ministry of Trade, Industry and Energy and Small and Medium Business Administration of the Republic of Korea [Project Management Number: S2090976; Project Name: Development of High-definition 3-dimensional R/F-compatible movable X-ray Imaging System with Detachable Function].

The method of controlling the rotation angle of a C-arm of the medical imaging apparatus according to an embodiment of the present invention may be implemented in the form of program instructions that can be executed by a variety of computer means, and may be stored in a computer-readable storage medium. The computer-readable storage medium may include program instructions, a data file, and a data structure solely or in combination. The program instructions that are stored in the medium may be designed and constructed particularly for the present invention, or may be known and available to those skilled in the field of computer software. Examples of the computer-readable storage medium include magnetic media such as a hard disk, a floppy disk and a magnetic tape, optical media such as CD-ROM and a DVD, magneto-optical media such as a floptical disk, and hardware devices particularly configured to store and execute program instructions such as ROM, RAM, and flash memory. Examples of the program instructions include not only machine language code that is constructed by a compiler but also high-level language code that can be executed by a computer using an interpreter or the like. The above-described hardware components may be configured to act as one or more software modules that perform the operation of the present invention, and vice versa.

Although the present invention has been described with reference to the specific details, such as the specific components, and the limited embodiments and drawings, this is provided merely to help a general understanding of the present invention, and is not intended to limit the present invention to the specific details and the embodiments and drawings. It will be apparent to those having ordinary knowledge in the art to which the present invention pertains that various modifications and variations can be made based on the above detailed description.

Therefore, the spirit of the present invention should not defined only by the disclosed embodiments, and not only the attached claims but also all equivalent to the claims and including equivalent modifications fall within the scope of the spirit of the present invention.

What is claimed is:

1. A method of controlling a medical imaging apparatus, comprising:
controlling, by a processor, a medical imaging apparatus to take a plurality of images including a region of interest of a subject, while a C-arm of the medical imaging apparatus rotates from a first rotation angle to a second rotation angle;
identifying, by the processor, a rotation angle of the C-arm of the medical imaging apparatus corresponding to each of the taken images;
storing, by the processor, the identified rotation angle, together with the taken image; and
extracting, by the processor, a rotation angle, stored along with an image selected as an optimal image from the taken images, as an optimal rotation angle.

2. The method of claim 1, further comprising:
controlling, by the processor, the C-arm to be placed at the extracted rotation angle.

3. The method of claim 1, further comprising:
providing, by the processor, a user menu which enables a user to select the optimal image from the taken images; and
receiving, by the processor, a selection input related to the optimal image via the user menu.

4. The method of claim 3, wherein the providing the user menu comprises, if at least one of the taken images is displayed on a screen, displaying, by the processor, a rotation angle stored along with the at least one image displayed on the screen, on the screen along with the at least one image displayed on the screen.

5. The method of claim 3, wherein the providing the user menu comprises:
extracting, by the processor, a contour line of a predetermined pattern with respect to the subject, from the at least one image displayed on a screen; and
displaying, by the processor, the extracted contour line to overlay on the at least one image displayed on the screen.

6. The method of claim 3, wherein the providing the user menu comprises:
determining, by the processor, at least one of optimal image candidate having highest suitability for the optimal image from the taken images based on a shape of a predetermined pattern with respect to the subject; and
displaying, by the processor, the determined at least one optimal image candidate on a screen prior to the other images.

7. The method of claim 1, further comprising:
selecting, by the processor, the optimal image from the taken images while considering a form in which the region of interest of the subject is represented on the image.

8. The method of claim 7, wherein selecting the optimal image comprises:
searching for, by the processor, an image taken in a direction perpendicular to the region of interest, from the taken images based on the shape as which the region of interest of the subject is represented; and
selecting, by the processor, the found image, taken in the direction perpendicular to the region of interest, as the optimal image.

9. A non-transitory computer-readable storage medium having stored therein program instructions, which when executed by a processor, causes the processor to:
control a medical imaging apparatus to take a plurality of images including a region of interest of a subject, while a C-arm of the medical imaging apparatus rotates from a first rotation angle to a second rotation angle;
detect a rotation angle of the C-arm of the medical imaging apparatus corresponding to each of the taken images;
store the detected rotation angle in a database, along with the taken image; and
extract a rotation angle, stored along with an image selected as an optimal image from the taken images, as an optimal rotation angle.

10. A device for controlling a medical imaging apparatus, comprising:
a processor configured to:
control a medical imaging apparatus to take a plurality of images including a region of interest of a subject, while a C-arm of the medical imaging apparatus rotates from a first rotation angle to a second rotation angle;
detect a rotation angle of the C-arm of the medical imaging apparatus corresponding to each of the taken images;
store the detected rotation angle in a database, along with the taken image; and
extract a rotation angle, stored along with an image selected as an optimal image from the taken images, as an optimal rotation angle.

11. The device of claim 10, the processor is further configured to:
provide a user menu that enables a user to select the optimal image from the taken images; and
receive a selection input related to the optimal image via the user menu.

12. The device of claim 10, the processor is further configured to:
search for an image taken in a direction perpendicular to the region of interest, from the taken images based on a shape as which the region of interest of the subject is represented; and
select the found image as the optimal image.
